(19) United States
(12) Patent Application Publication
(10) Pub. No.: US 2021/0090708 A1
(43) Pub. Date: Mar. 25, 2021

(54) SYSTEM AND METHOD TO OFFER WELLNESS PROGRAMS

(71) Applicant: Creative Choice Inc., Palm Beach Gardens, FL (US)

(72) Inventors: Dilip Barot, Palm Beach Gardens, FL (US); Rashmin Barot, Gandhinagar (IN)

(21) Appl. No.: 16/580,227

(22) Filed: Sep. 24, 2019

(51) Int. Cl. G16H 20/30; G16H 20/70; G16H 20/90

(52) U.S. Cl. CPC G16H 20/30; G16H 20/90; G16H 20/70

(57) ABSTRACT

A system and method to offer wellness programs are provided. The system includes a plurality of designated geographical areas configured to impart wellness of body, mind and spirit by stimulating sight, smell, sound, taste and touch. The system also includes wearable devise(s) configured to capture biophysical parameters of the user; a user database subsystem configured to receive registration details from the user, maintain a list of support staff, and provide list of available offerings; a user-staff interaction subsystem configured to enable seamless communication between selected support staff and the user; an evaluation subsystem configured to evaluate a level of wellness of the user; a score generation subsystem configured to generate a wellness score to each of the user; a customisation subsystem configured to receive the feedback from the user and the captured biophysical parameters of the user, and customise the wellness program specific to the requirement of individual user.

### FIELD OF INVENTION

Embodiments of a present disclosure relate to providing health and wellness and more particularly to system and method to offer wellness programs to one or more users.

### BACKGROUND

Health and wellness are the most important assets a person can have; however, it has become elusive for most of us because of our ways of living. Current globalization and ever-increasing demands for greater productivity have led to excessive stress in our professional and personal lives. More and more people are looking for ways to find balance in their fast-paced lives. Even people enjoying comfortable life, in physical sense, are also falling sick for plethora of reasons. People are facing issues like high blood pressure, heart problems, obesity, headaches, depression and anxiety, Gastrointestinal problems, and accelerated ageing.

More and more people are looking for ways to find balance in their fast-paced lives. People are resorting to medication of different sorts for treating lifestyle related illnesses. However, it has been proven difficult to achieve wellness which is more than being free from illness, it is a dynamic process of change and growth.

There has been a recent emphasis on wellness, and various programs have been devised for helping people in achieving wellness, however none of them have identified the five pillars of wellness (mindfulness, relaxation, sleep, exercise, and nutrition) nor have they integrated the programs through custom applications allowing participants to monitor their progress and enjoy direct access to their lifestyle coach on a continuing basis. Therefore, such programs have not been able to provide the desired outcomes.

Hence, there is a need for an improved system and method to offer wellness programs for healthful living to address the aforementioned issues.

### BRIEF DESCRIPTION

In accordance with an embodiment of the present disclosure, a system and method to offer wellness programs for healthful living is provided. The system includes a centralized touch screen smart console housed within each of a residential unit in a plurality of designated geographical areas. The centralised touch screen smart console is configured to display parameters of various devices and IoT devices configured in the residential unit, to operate the various devices and the IoT devices, to interact with one or more support staff, and to provide feedback regarding the wellness program. The system also includes a plurality of indoor and outdoor meditation units for imparting mental relaxation. The system also includes one or more massage units for imparting muscular and sensory relaxation to the one or more users by stimulating release of endorphins and dopamine in body of the one or more users. The system also includes means of imparting hydrotherapy comprising jacuzzi, spa, LED sauna, showers, mud hut, and labyrinth, pools for stimulating release of endorphins and dopamine in body of the one or more users. The system also includes means of imparting aromatherapy operatively coupled with the means of imparting hydrotherapy and the one or more massage units. The system also includes one or more physical workout units provided with a plurality of fitness equipment for stimulating release of endorphins, adrenaline, serotonin and dopamine in body of the one or more users. The system also includes one or more wearable devises configured to capture data representative of biophysical parameters of the one or more users. The system also includes a user database subsystem configured to receive registration details from the one or more users to register the one or more users. The user database subsystem is also configured to maintain a list of support staff along with a corresponding specialisation list of each of the support staff. The user database subsystem is also configured to provide a list of available lightings and colours; available aromas, flowers, aroma diffusers or candles; at least one of sounds, music, and playlists; at least one of fruits, food or beverages, and types of fabrics with the residential unit in a plurality of designated geographical areas. The system also includes a user-staff interaction subsystem configured to enable seamless communication between selected one or more support staff and the corresponding one or more users via a smart device associated with the one or more users. The system also includes an evaluation subsystem configured to evaluate a level of wellness of the corresponding one or more users upon receiving a plurality of wellness history from the one or more users. The system also includes a score generation subsystem configured to generate a wellness score to each of the one or more users based on an evaluation result. The system also includes a customisation subsystem configured to receive a feedback from the one or more users and a captured data representative of biophysical parameters of the corresponding one or more users. The customisation subsystem is also configured to customise the wellness program based on the received feedback and captured data representative of biophysical parameters of the one or more users. The system also includes a training subsystem configured to schedule at least one training session for each of the one or more users based on a generated score to improve the wellness of the corresponding one or more users.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF THE DRAWINGS

The disclosure will be described and explained with additional specificity and detail with the accompanying figures in which:

FIG. 1 is a block diagram representation of a system to offer wellness programs to one or more users in accordance with an embodiment of the present disclosure;

FIG. 2 is a block diagram of a wellness computer or a server in accordance with an embodiment of the present disclosure; and

FIG. 3 is a flow chart representing steps involved in a method for offering wellness programs to one or more users in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure.

The terms “comprises”. “comprising”, or any other variations thereof, are intended to cover a non-exclusive inclusion, such that a process or method that comprises a list of steps does not include only those steps but may include other steps not expressly listed or inherent to such a process or method. Similarly, one or more devices or sub-systems or elements or structures or components preceded by “comprises . . . a” does not, without more constraints, preclude the existence of other devices, sub-systems, elements, structures, components, additional devices, additional sub-systems, additional elements, additional structures or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

Embodiments of the present disclosure relate to a system and method to offer wellness programs to one or more users.

The object of the present invention is to provide a comprehensive wellness program to maximize the ability of people to reach and sustain a position of maximum health and wellness from an emotional, mental, and physical standpoint. It is complimented by applications that monitor progress toward established goals and allow for continuing feedback and access to a continuing program of support.

The presently disclosed wellness program is the only program that identifies and defines five pillars for health and wellness (mindfulness, relaxation, sleep, exercise, and nutrition) and provides programs within the overall program to address each pillar individually. It also allows participants to have continuing access to support from staff experts when they are not at the health and wellness centre. The wellness program comprises many fitness programs, multiple diet programs, and other assist programs that address individual facets of health and wellness, while allowing for continuous monitoring, support, and updating on an individual basis.

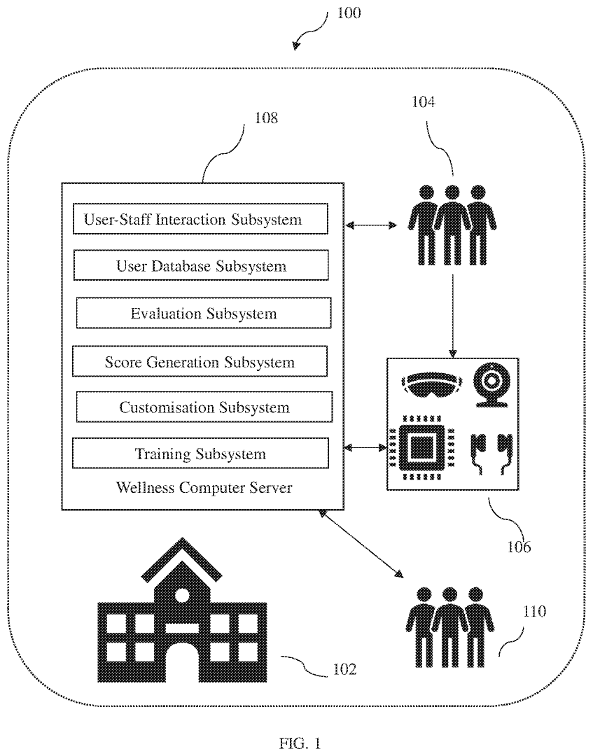

FIG. 1 is a block diagram representation of a system 100 to offer wellness programs to one or more users in accordance with an embodiment of the present disclosure. The system includes a centralized touch screen smart console housed within each of a residential unit in a plurality of designated geographical areas 102. The plurality of designated geographical areas 102 is configured to impart wellness of body, mind and spirit of the one or more users by stimulating sight, smell, sound, taste and touch. The plurality of designated geographical areas 102 is configured to have residential units for accommodating one or more registered users 104. The residential units are configured with various devices and IoT devices 106 for automation, and controlling temperature and humidity, and communication etc. Each of the residential units being provided with a centralized touch screen smart console (not shown in FIG. 1). The centralized touch screen smart console is a smart device operatively coupled with the wellness computer server 108, and handheld devices associated with the registered users 104 and support staff 110

In one embodiment, the centralised touch screen smart console which may be a smart device which may be operatively coupled to the wellness computer server 108. In another embodiment, the centralised touch screen smart console may be a handheld device associated with corresponding one or more registered users 104 and one or more support staff 110. In one exemplary embodiment, the one or more support staff 110 may include guides, instructors, massage providers, therapists and caregivers.

Furthermore, the centralised touch screen smart console is configured to display parameters of various devices and IoT devices 106 configured in the residential unit, to operate the various devices and the IoT devices 106, to interact with the one or more support staff 110, and to provide feedback regarding the wellness program. In one embodiment, the centralised touch screen smart console may be configured to operate the various devices and the IoT devices 106 based on the input which may be received from the one or more users or the one or more support staff 110 via centralized touch screen smart console and/or handheld devices associated with the one or more registered users 104 and/or support staff 110. Therefore, all the parameters may be controlled and changed remotely.

In one exemplary embodiment, the centralized touch screen smart console may also be configured to enable interaction of the one or more users with the corresponding one or more support staff 110 via text, audio and video or any combination thereof.

The system also includes a plurality of indoor and outdoor meditation units for imparting mental relaxation. The meditation units may be referred to as meditation centres with optimum conditions and trained staff to assist the one or more users. This may help in bringing down the levels of stress, anxiety and depression.

The system also includes one or more massage units for imparting muscular and sensory relaxation to a user. The massage so provided by trained professionals stimulates release of endorphins and dopamine in body of the user imparting a sense of pleasure and calmness.

Furthermore, the system includes means of imparting hydrotherapy comprising such as, but not limited to jacuzzi, spa, LED sauna, showers, mud hut, and labyrinth, pools for stimulating release of endorphins and dopamine in body of the one or more users. The hydrotherapy brings down body temperature level to optimum and stimulates release of endorphins and dopamine in body of the user imparting a calming effect. The aforementioned means of imparting hydrotherapy may be configured within dedicated areas.

The system also includes means of imparting aromatherapy operatively coupled with the means of imparting hydrotherapy and the one or more massage units. The aromatherapy may be implemented through inhalation or topical application. During the inhalation, one or more essential oils being evaporated into air using one of a diffuser container, spray, or oil droplets. However, during the topical application at least one of a one or more message oils and skin care products are enabled to be absorbed through skin during the topical application by the massage, thereby increasing absorption and boosting blood circulation.

The system also includes one or more physical workout units provided with a plurality of fitness equipment for stimulating release of endorphins, adrenaline, serotonin and dopamine in body of the one or more users. The designed workout routine or fitness classes helps in release of endorphins, adrenaline, serotonin and dopamine in body of the one or more users.

The system also includes one or more wearable devises configured to capture data representative of biophysical parameters of the one or more users. In one embodiment, the one or more wearable devices may be a wrist band, a head band, a locket, eye wear such as goggles, and a wearable fabric. In one exemplary embodiment, the biophysical parameters of the one or more users may include blood pressure, heartbeat, temperature, breathing rate, hydration level, and trans-epidermal water loss.

Furthermore, the system includes a user database subsystem operatively coupled to the centralized touch screen smart console. The user database is configured to receive registration details from the one or more users to register the one or more users. In one embodiment, the registration details of the one or more users may include name, address, contact details, duration of stay, any specific health issue(s), and preferences regarding lighting, colours, aromas, food, music, and beverages etc. This enables the user's flexibility to select his/her preference while registering or during their stay in the residential unit.

The user database subsystem is also configured to maintain a list of support staff 110 along with a corresponding specialisation list of each of the one or more support staff 110. In one embodiment, the user database subsystem may also be configured to update the list of support staff 110. Further, the user database subsystem is also configured to provide a list of available lightings and colours; available aromas, flowers, aroma diffusers or candles; at least one of sounds, music, and playlists; at least one of fruits, food or beverages, and types of fabrics with the residential unit in a plurality of designated geographical areas 102.

The system also includes a user-staff interaction subsystem configured to enable seamless communication between selected one or more support staff 110 and the corresponding one or more users via a smart device associated with the one or more users. This helps in providing real-time assistance to users and monitoring the progress of the users even when they are not in residing the plurality of designated geographical areas 102.

Furthermore, the system includes an evaluation subsystem. The evaluation subsystem is configured to evaluate a level of wellness of the corresponding one or more users upon receiving a plurality of wellness history from the one or more users. In one embodiment, the plurality of wellness history associated with the corresponding one or more users may be received during the process of registration. As used herein, the term “wellness history” is defined as the bodily wellness of the corresponding one or more users in the past prior to registering on the platform. In one exemplary embodiment, the level of wellness of the corresponding one or more users may be calculated by mapping the plurality of wellness history with a pre-defined set of data.

The system also includes a score generation subsystem configured to generate a wellness score to each of the one or more users based on an evaluation result, wherein the wellness score is generated upon comparing the evaluation result with a pre-defined set of wellness score, wherein the predefined set of wellness score is stored in the database.

The system also includes a customisation subsystem configured to receive a feedback from the one or more users and a captured data representative of biophysical parameters of the corresponding one or more users. The customisation subsystem is also configured to customise the wellness program based on the received feedback and captured data representative of biophysical parameters of the one or more users to optimizes the effectiveness of the wellness program specific to the one or more users. In one exemplary embodiment, the system may further include a feedback subsystem which may be configured to receive one or more feedbacks from the corresponding one or more users based on the wellness programs. In such embodiment, the feedback may include preferred lighting and colour (sense of sight); preferred aroma, flowers, aroma diffuser or candles (sense of smell); preferred sound or music (sense of sound); preferred fruits, food or beverage (sense of taste); and preferred softness of fabric to be used (sense of touch). The lighting is meant for the luminosity and colour of lights used in the residential units. The colour is meant for colour of the fabric to be used such as throws, pillows, bedsheets, etc. The preferred softness of the fabric means hard/soft and down or foam pillow, light cotton blanket or heavier comforter, lightweight robe, or heavier plush robe. The predefined weightages have been assigned to each of the received feedback and biophysical parameters of the user for the purposes of customisation.

Furthermore, the system includes a training subsystem configured to schedule at least one training session for each of the one or more users based on a generated score to improve the wellness of the corresponding one or more users. In one embodiment, the training subsystem may also be configured to schedule at least one group training session for the one or more users based on a generated score to improve the wellness of the one or more users. In such embodiment, the training session may be scheduled in order to guide the one or more users to take proper wellness programs. The training session may be scheduled and suggested based on the wellness score generated for the corresponding one or more users.

In one specific embodiment, the one or more users may be issued with at least one of a health insurance program or a wellness program in order to optimize the effectiveness of the wellness program specific to the one or more users.

Furthermore, in one exemplary embodiment, the system may be integrated with at least one a virtual reality device, an augmented reality device or a mixed reality device in order to provide a sophisticated experience of the wellness program provided to the corresponding one or more users.

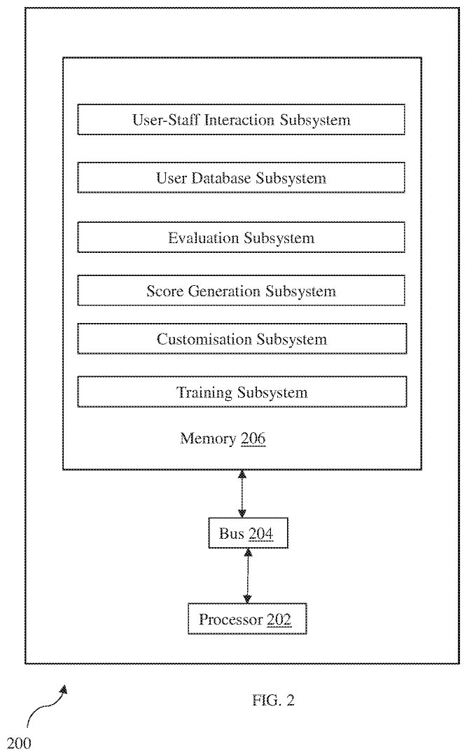

FIG. 2 is a block diagram of a wellness computer or server 200 in accordance with an embodiment of the present disclosure. The wellness server 200 includes processor(s) 202, and memory 206 coupled to the bus 204. As used herein, the processor and memory are substantially similar to wellness computer server 108 of the system 100 of FIG. 1

The processor(s), as used herein, means any type of computational circuit, such as, but not limited to, a microprocessor, a microcontroller, a complex instruction set computing microprocessor, a reduced instruction set computing microprocessor, a very long instruction word microprocessor, an explicitly parallel instruction computing microprocessor, a digital signal processor, or any other type of processing circuit, or a combination thereof.

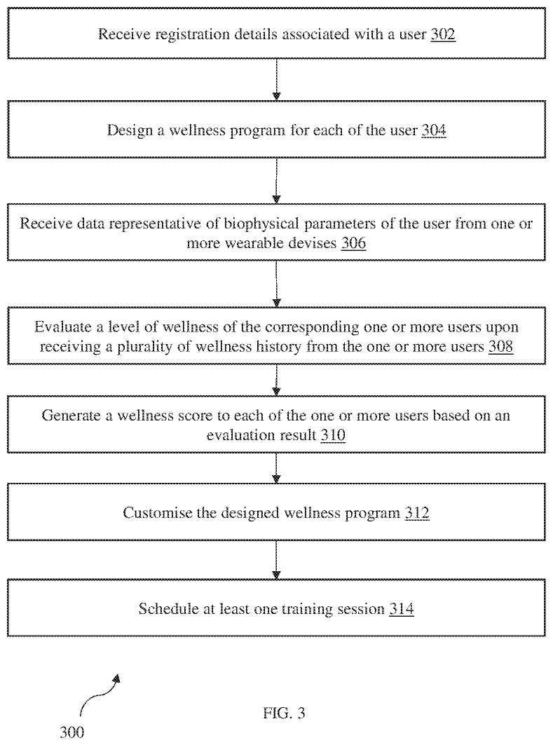

The memory 206 includes a plurality of subsystems stored in the form of executable program which instructs the processor 202 to perform method steps illustrated in FIG. 3. The memory 206 has following subsystems: a user database subsystem, a user-staff interaction subsystem, an evaluation subsystem, a score generation subsystem, a customisation subsystem and a training subsystem.

The user database subsystem is configured to receive registration details from the one or more users to register the one or more users and to, to maintain a list of support staff along with a corresponding specialisation list of each of the support staff and to provide a list of available lightings and colours; available aromas, flowers, aroma diffusers or candles; at least one of sounds, music, and playlists; at least one of fruits, food or beverages, and types of fabrics with the residential unit in a plurality of designated geographical areas.

The user-staff interaction subsystem is configured to enable seamless communication between selected one or more support staff and the corresponding one or more users via a smart device associated with the one or more users.

The evaluation subsystem is configured to evaluate a level of wellness of the corresponding one or more users upon receiving a plurality of wellness history from the one or more users.

The score generation subsystem is configured to generate a wellness score to each of the one or more users based on an evaluation result, wherein the wellness score is generated upon comparing the evaluation result with a predefined set of wellness score, wherein the predefined set of wellness score is stored in a database.

The customisation subsystem is configured to receive a feedback from the one or more users and a captured data representative of biophysical parameters of the corresponding one or more users and to customise the wellness program based on the received feedback and captured data representative of biophysical parameters of the one or more users.

The training subsystem is configured to schedule at least one training session for each of the one or more users based on a generated score to improve the wellness of the corresponding one or more users.

Computer memory elements may include any suitable memory device(s) for storing data and executable program, such as read only memory, random access memory, erasable programmable read only memory, electrically erasable programmable read only memory, hard drive, removable media drive for handling memory cards and the like. Embodiments of the present subject matter may be implemented in conjunction with program modules, including functions, procedures, data structures, and application programs, for performing tasks, or defining abstract data types or low-level hardware contexts. Executable program stored on any of the above-mentioned storage media may be executable by the processor(s).

FIG. 3 is a flow chart representing steps involved in a method 300 for offering wellness programs to one or more users in accordance with an embodiment of the present disclosure. The method 300 includes receiving registration details from the one or more users in step 302. In one embodiment, receiving the registration details may include receiving include name, address, contact details, duration of stay, any specific health issue(s), and preferences regarding lighting, colours, aromas, food, music, beverages and the like. This enables the user's flexibility to select his/her preference while registering or during their stay. The registration details are received at the wellness computer server via computing devices associated with the one or more user and stored in the user database subsystem.

The method 300 also includes designing a wellness program for each of the one or more users in step 304, wherein the wellness program includes scheduling for meditation in a plurality of indoor and outdoor meditation units for mental relaxation, one or more massage therapy sessions in one or more massage units for imparting muscular and sensory relaxation to the one or more users by stimulating release of endorphins and dopamine in body of the one or more users, one or more hydrotherapy sessions, one or more aromatherapy sessions, and one or more physical workout sessions for the corresponding one or more users.

Furthermore, the method 300 includes receiving data representative of biophysical parameters of the one or more users from one or more wearable devises in step 306. In one embodiment, receiving the data representative of biophysical parameters may include receiving blood pressure, heartbeat, temperature, breathing rate, hydration level, trans-epidermal water loss or a combination thereof. The data representative of biophysical parameters of the one or more users being captured via one or more wearable devises put on the corresponding one or more users. The wearable devices in the present context may be a wrist band, a head band, a locket, eye wear such as goggles, and a wearable fabric or dress.

The method 300 also includes evaluating a level of wellness of the corresponding one or more users upon receiving a plurality of wellness history from the one or more users in step 308. In one embodiment, evaluating the level of wellness of the one or more corresponding users may include evaluating the level of wellness of the corresponding one or more users by mapping the plurality of wellness history with a pre-defined set of data. In one embodiment, evaluating the level of wellness of the corresponding one or more users by an evaluation subsystem.

The method 300 also includes generating a wellness score to each of the one or more users based on an evaluation result in step 310. The wellness score is generated upon comparing the evaluation result with a predefined set of wellness data, wherein the predefined set of wellness data is stored in a database. In one embodiment, generating the wellness score by a score generation subsystem.

Furthermore, the method 300 also includes customising the designed wellness program based on received a feedback from the one or more users regarding preferred lighting and colour, preferred aroma, flowers, aroma diffuser or candles, preferred sound or music, preferred fruits, food or beverage, preferred softness of fabric to be used during the wellness program in step 312. In one embodiment, customising the designed wellness program may include customising the designed wellness program by a customisation subsystem.

The method 300 also includes scheduling at least one training session for each of the one or more users based on a generated score to improve the wellness of the corresponding one or more users in step 314. In one embodiment, scheduling the at least one training session may include scheduling the at least one training session by a training subsystem. In one exemplary embodiment, the method 300 may further include scheduling at least one group training session for the one or more users based on a generated score to improve the wellness of the one or more users. In such embodiment, scheduling the at least one group training session may include scheduling the at least one training session by the training subsystem.

In one specific embodiment, the method 300 may further include issuing the one or more users with at least one of a health insurance program or a wellness program in order to optimize the effectiveness of the wellness program specific to the one or more users.

In another specific embodiment, the method 300 may include integrating the residential unit with at least one a virtual reality device, an augmented reality device or a mixed reality device in order to provide a sophisticated experience of the wellness program provided to the corresponding one or more users.

Various embodiments of the system and method for customising and offering wellness programs for healthful living described above enables the users to achieve desired health and wellness in a holistic way. The present invention combines nature, architecture, philosophy, technology, and Eastem wellness, with all of its knowledge of the health of the body, mind, and spirit. This may be really helpful in bringing down the stress, anxiety and depression related diseases in fast paced lives of people.

It will be understood by those skilled in the art that the foregoing general description and the following detailed description are exemplary and explanatory of the disclosure and are not intended to be restrictive thereof.

While specific language has been used to describe the disclosure, any limitations arising on account of the same are not intended. As would be apparent to a person skilled in the art, various working modifications may be made to the method in order to implement the inventive concept as taught herein.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, order of processes described herein may be changed and are not limited to the manner described herein. Moreover, the actions of any flow diagram need not be implemented in the order shown; nor do all of the acts need to be necessarily performed. Also, those acts that are not dependent on other acts may be performed in parallel with the other acts. The scope of embodiments is by no means limited by these specific examples.

We claim:

1. A system to offer wellness program to one or more users comprising:
a centralized touch screen smart console housed within each of a residential unit in a plurality of designated geographical areas, wherein the plurality of designated geographical areas are configured to impart wellness of body, mind and spirit of the one or more users by stimulating sight, smell, sound, taste and touch,
wherein the centralised touch screen smart console is configured to display parameters of various devices and IoT devices configured in the residential unit, to operate the various devices and the IoT devices, to interact with one or more support staff, and to provide feedback regarding the wellness program;
a plurality of indoor and outdoor meditation units for imparting mental relaxation;
one or more massage units for imparting muscular and sensory relaxation to the one or more users by stimulating release of endorphins and dopamine in body of the one or more users;
means of imparting hydrotherapy comprising jacuzzi, spa, LED sauna, showers, mud hut, and labyrinth, pools for stimulating release of endorphins and dopamine in body of the one or more users;
means of imparting aromatherapy operatively coupled with the means of imparting hydrotherapy and the one or more massage units;
one or more physical workout units provided with a plurality of fitness equipment for stimulating release of endorphins, adrenaline, serotonin and dopamine in body of the one or more users;
one or more wearable devises configured to capture data representative of biophysical parameters of the one or more users;
a user database subsystem operatively coupled to the centralized touch screen smart console, and operable by one or more processors, wherein the user database is configured to:
receive registration details from the one or more users to register the one or more users;
maintain a list of support staff along with a corresponding specialisation list of each of the support staff;
provide a list of available lightings and colours; available aromas, flowers, aroma diffusers or candles; at least one of sounds, music, and playlists; at least one of fruits, food or beverages, and types of fabrics with the residential unit in a plurality of designated geographical areas;
a user-staff interaction subsystem operable by the one or more processors, and configured to enable seamless communication between selected one or more support staff and the corresponding one or more users via a smart device associated with the one or more users;
an evaluation subsystem operable by the one or more processors, and configured to evaluate a level of wellness of the corresponding one or more users upon receiving a plurality of wellness history from the one or more users;
a score generation subsystem operable by the one or more processors, and configured to generate a wellness score to each of the one or more users based on an evaluation result, wherein the wellness score is generated upon comparing the evaluation result with a predefined set of wellness data, wherein the predefined set of wellness data is stored in a database;
a customisation subsystem operable by the one or more processors, and configured to:
receive a feedback from the one or more users and a captured data representative of biophysical parameters of the corresponding one or more users;
customise the wellness program based on the received feedback and captured data representative of biophysical parameters of the one or more users; and
a training subsystem operable by the one or more processors, and configured to schedule at least one training session for each of the one or more users based on a generated score to improve the wellness of the corresponding one or more users.

2. The system as claimed in claim 1, wherein the aromatherapy being implemented through one of an inhalation and a topical application.

3. The system as claimed in claim 2, wherein one or more essential oils being evaporated into air using one of a diffuser container, spray, or oil droplets during the inhalation.

4. The system as claimed in claim 2, wherein at least one of a one or more message oils and skin care products are enabled to be absorbed through skin during the topical application by the massage, thereby increasing absorption and boosting blood circulation.

5. The system as claimed in claim 1, wherein the biophysical parameters of the user comprises blood pressure, heart beat, temperature, breathing rate, hydration level, and trans-epidermal water loss.

6. The system as claimed in claim 1, wherein the feedback comprises preferred lighting and colour; preferred aroma, flowers, aroma diffuser or candles; preferred sound or music; preferred fruits, food or beverage, preferred softness of fabric used.

7. The system as claimed in claim 1, wherein the training subsystem is further configured to schedule at least one group training session for the one or more users based on a generated score to improve the wellness of the one or more users.

8. A method for offering wellness program to one or more users comprising:
receiving, by a user database subsystem, registration details from the one or more users;
designing a wellness program for each of the one or more users, wherein the wellness program includes scheduling for meditation in a plurality of indoor and outdoor meditation units for mental relaxation, one or more massage therapy sessions in one or more massage units for imparting muscular and sensory relaxation to a user by stimulating release of endorphins and dopamine in body of the one or more users, one or more hydrotherapy sessions, one or more aromatherapy sessions, and one or more physical workout sessions for the corresponding one or more users;
receiving, by one or more wearable devices, data representative of biophysical parameters of the user from one or more wearable devises;
evaluating, by an evaluation subsystem, a level of wellness of the corresponding one or more users upon receiving a plurality of wellness history from the one or more users;
generating, by a score generation subsystem, a wellness score to each of the one or more users based on an evaluation result, wherein the wellness score is generated upon comparing the evaluation result with a predefined set of wellness data, wherein the predefined set of wellness data is stored in a database;
customising, by a customisation subsystem, the designed wellness program based on received a feedback from the one or more users regarding preferred lighting and colour, preferred aroma, flowers, aroma diffuser or candles, preferred sound or music, preferred fruits, food or beverage, preferred softness of fabric to be used during the wellness program; and
scheduling, by a training subsystem, at least one training session for each of the one or more users based on a generated score to improve the wellness of the corresponding one or more users.

9. The method as claimed in claim 8, wherein receiving the data representative of biophysical parameters of the user from one or more wearable devises comprises receiving the data representative of at least one of blood pressure, heart beat, temperature, breathing rate, hydration level, trans-epidermal water loss or a combination thereof.

10. The method as claimed in claim 8, comprising scheduling, by the training subsystem, at least one group training session for the one or more users based on a generated score to improve the wellness of the one or more users.
